(19) United States
(12) Patent Application Publication
(10) Pub. No.: US 2009/0175980 A1
(43) Pub. Date: Jul. 9, 2009

(54) WELLNESS BASED PET DIETS THAT TAKE INTO CONSIDERATION BREED TYPE AND GENETIC PREDISPOSITION OF A PET

(75) Inventors: Neil Willcocks, Brentwood, TN (US); Tiffany L. Bierer, Brentwood, TN (US)

Correspondence Address: FITZPATRICK CELLA HARPER & SCINTO, 30 ROCKEFELLER PLAZA, NEW YORK, NY, 10112, US

(73) Assignee: MARS, INCORPORATED, McLean, VA (US)

(21) Appl. No.: 12/336,258

(22) Filed: Dec. 16, 2008

Related U.S. Application Data
(60) Provisional application No. 61/014,318, filed on Dec. 17, 2007.

(51) Int. Cl. A23K 1/18; C12Q 1/68

(52) U.S. Cl. 426/2; 435/6

(57) ABSTRACT

In order to improve or manage a pet's wellness and health, and promote longevity and a healthy vitality, the pet's condition is assessed. A recommended diet regimen is determined that takes into account the pet's breed type(s), genetic predisposition to a disease or disorder, lifestyle, and pet feeding preferences of the pet's owner. The owner is provided with at least one option that satisfies the recommended diet regimen.

### CROSS-REFERENCE TO RELATED APPLICATIONS

The present application claims benefit of U.S. Provisional Patent Application No. 61/014,318, filed on Dec. 17, 2007, the entire disclosure of which is incorporated by reference herein.

### BACKGROUND OF THE INVENTION

1. Field of the Invention

The present invention relates to a system and a method for promoting the longevity of a pet through a healthy diet regimen that benefits both the pet as well as the pet's owner. Through an assessment of the pet's current health status, a determination is made that a change in the pet's diet regimen would improve or manage the pet's wellness and health, and promote a longer life span and a healthier vitality. More specifically, a recommended diet regimen is determined from the assessment, and the owner is provided with at least one option that satisfies the recommended diet regimen. For example, the recommended diet regimen is determined based on an assessment of a breed type or a combination of breed types in the dog's background, genetic predisposition to a disease or disorder, the pet's size, body-fat content, weight, coat condition, sex, life stage, activity level, overall health, and other characteristics indicative of the general well-being of the pet, as well as an assessment of the lifestyle of the pet and the pet feeding preferences of the pet's owner.

2. Related Art

Pet owners generally want to provide the best nutrition for their pets, but often are either constrained by a limited budget or not aware of dietary needs that are best suited for their pets. As a result, pet owners often opt to purchase premium foods and treats while foregoing supplements in order to stay within their budgets. Pet owners also have a tendency to think that by providing premium foods and treats to their pets, they are fulfilling their pets' dietary needs. However, most of the time, this is not the case; for pets that should be receiving supplements to improve their coats, or for pets that have medical conditions that should be treated with pharmaceuticals, for example, this option is not the best course of action for the well-being of the pets.

On the other hand, for pet owners that are not constrained by pet budgets, their pets often are spoiled by an overabundance of food and high-calorie treats, in the mistaken belief that extra food means extra love, which ultimately results in unhealthy, overweight pets. Therefore, these pets may not be receiving foods, supplements, and/or pharmaceuticals that are particularly suited for their conditions. In other words, despite the best intentions of these pet owners, they may not be the most qualified people to determine the best diets for their pets.

Pet owners may have special feeding relationships with their pets, in which the owners have a preferred feeding schedule that is not well suited for the typical container sizes of pet foods offered today. For example, an owner may prefer to feed her cat five or more times per day to coincide with the times when the owner herself eats.

Many pet owners are uncertain as to the quantity or type of foods (wet and/or dry), treats, supplements, and the like, to give to their pets. As a result of this uncertainty the owners may tend to overfeed their pets or to feed their pets with a type of food that is less than optimal (based on the current conditions of the pets). For example, a sedentary and overweight pet should be receiving low-calorie foods and treats in order to minimize weight gain. However, the lack of variety of low-calorie pet foods has resulted in an unhappy feeding experience for the pet owners, because they cannot vary the types and flavors of food given to their pets without going off the low-calorie diet.

Further, different types of pets may have different dietary needs, which may be beyond the scope of knowledge of most pet owners. In addition, many pets have genetic predispositions to different diseases or disorders of which the pet owners are not aware.

Given the foregoing, a need exists for a convenient way for a pet owner to enhance the health and vitality of her pet through a diet regimen, taking into account the breed(s) and/or the genetic predisposition of the pet, that improves or manages the pet's wellness and health and that promotes a longer life span and a healthier vitality without compromising on the quantity of food given to the pet and without compromising the diversity in the types and flavors of the food given to the pet.

### BRIEF DESCRIPTION OF THE INVENTION

According to an embodiment of the present invention, a method of promoting longevity and a healthy vitality of a pet is provided. The method includes performing an assessment of a condition of the pet, wherein the assessment takes into consideration a breed type of the pet determined by a DNA-based test, determining a diet regimen for improving or managing the condition of the pet, based on the assessment, and providing at least one option for satisfying the diet regimen.

In an aspect of the embodiment, the assessment takes into consideration a lifestyle of the pet. For example, the lifestyle of the pet includes at least one of: an activity level of the pet, a type of exercise performed by the pet, whether the pet is allowed outdoors, whether the pet is sedentary, whether the pet is handicapped, and whether one or more other pets cohabit with the pet.

In another aspect of the embodiment, the assessment takes into consideration pet feeding preferences of the pet's owner. For example, the pet feeding preferences may include any one or more of: feeding frequency, type of food, type of treat, forbidden foods, and portion size per feeding.

In a further aspect of the embodiment, the assessment includes determining a genetic predisposition of the pet for a disease or disorder. For example, the genetic predisposition of the pet to the disease or disorder may be determined by examining a genetic marker associated with the disease or disorder. The diet regimen may be tailored to prevent, manage, delay manifestation, treat, or reduce effects associated with the disease or disorder to which the pet is genetically predisposed.

In another aspect of the embodiment, the assessment takes into consideration at least one physical condition or attribute of the pet, the physical condition or attribute including body-fat content, size, weight, life stage, sex, coat condition, skin condition, body type, health of the pet, or any combination thereof.

In a further aspect of the embodiment, the assessment includes an exercise test (e.g., a treadmill test). For example, the exercise test may be a test that enables a determination of a heart rate of the pet during a period of activity and a heart rate during a period of inactivity. In another example, the exercise test may be a test that enables a determination of a level of exercise the pet can endure.

In another aspect of the embodiment, the diet regimen includes a recommended daily calorie intake for the pet and/or a recommended quantity of specific foods and treats.

In yet another aspect of the embodiment, the at least one option may include an option for one or more of: a natural (AAFCO) product, an organic product, a brand type, a flavor type, a budget level, a nutritional content, a texture type, a type of filler, a vegetarian product, and a food format.

In a further aspect of the embodiment, the at least one option may include any combination of: food, treats, supplements, and pharmaceuticals. For example, the food may be wet food, dry food, or a combination thereof.

In another aspect of the embodiment, the assessment may be performed using a computerized survey completed by a veterinarian, by a trained consultant, or by a pet owner. The computerized survey may be obtained from a computer-readable storage medium or a server accessible via the Internet. For example, the computer-readable storage medium may be a CD or a DVD.

In still another aspect of the embodiment, the method further includes performing a plurality of assessments of the pet over a period of time during which the pet is on the diet regimen, and refining the diet regimen based on the plurality of assessments.

According to another embodiment of the present invention, a method of promoting longevity and a healthy vitality of a mixed-breed pet is provided. The method includes determining two or more breed types of the pet, based on a DNA-based test, identifying a genetic predisposition for a disease or disorder of a breed type determined based on the DNA-based test, and examining a genetic marker associated with the disease or disorder to determine whether the pet is genetically predisposed to the disease or disorder. For example, the genetic marker may be obtained from a bodily fluid or a tissue sample of the pet.

In an aspect of the embodiment, the method further includes determining a diet regimen for the pet, and providing at least one option for satisfying the diet regimen. For example, the diet regimen may be tailored to prevent, manage, delay manifestation, treat, or reduce effects associated with the disease or disorder. In another example, the diet regimen may include any combination of: food, treats, supplements, and pharmaceuticals.

In another aspect of the embodiment, the method further includes performing a plurality of assessments of a condition of the pet over a period of time during which the pet is on the diet regimen, and refining the diet regimen based on the plurality of assessments.

In a further aspect of the embodiment, the DNA-based test identifies at least a dominant or major breed type of the pet or at least a subordinate or minor breed type of the pet or a combination thereof. In this aspect, the method may further include determining a diet regimen tailored to benefit a dominant or major breed of the pet, tailored to benefit a subordinate or minor breed of the pet, or that includes a plurality of dietary items which include at least a first dietary item tailored to benefit a dominant or major breed of the pet and a second dietary item tailored to benefit a subordinate or minor breed of the pet.

According to another embodiment of the present invention, a method of promoting longevity and a healthy vitality of a mixed-breed pet is provided. The method includes performing an assessment of a condition of the pet, wherein the assessment takes into consideration at least one breed type of the pet determined by a DNA-based test, determining a diet regimen for improving or managing the condition of the pet, based on the assessment, and providing at least one option for satisfying the diet regimen.

In an aspect of the embodiment, the assessment takes into consideration a lifestyle of the pet. For example, the lifestyle of the pet may include at least one of: an activity level of the pet, a type of exercise performed by the pet, whether the pet is allowed outdoors, whether the pet is sedentary, whether the pet is handicapped, and whether one or more other pets cohabit with the pet.

In another aspect of the embodiment, the assessment takes into consideration pet feeding preferences of the pet's owner. For example, the pet feeding preferences may include any one or more of: feeding frequency, type of food, type of treat, forbidden foods, and portion size per feeding.

In a further aspect of the embodiment, the DNA-based test identifies at least one dominant or major breed type of the pet or at least one subordinate or minor breed type of the pet or a combination thereof. In this aspect, the diet regimen may be tailored to benefit a dominant or major breed of the pet or to benefit the subordinate or minor breed of the pet, or the diet regimen may include a plurality of dietary items which include at least a first dietary item tailored to benefit a dominant or major breed of the pet and a second dietary item tailored to benefit a subordinate or minor breed of the pet.

In another aspect of the embodiment, the assessment includes determining a genetic predisposition of the pet for a disease or disorder. For example, the genetic predisposition of the pet to the disease or disorder may be determined by examining a genetic marker associated with the disease or disorder. The diet regimen may be tailored to prevent, manage, delay manifestation, treat, or reduce effects associated with the disease or disorder to which the pet is genetically predisposed.

In a further aspect of the embodiment, the assessment takes into consideration at least one physical condition or attribute of the pet, the physical condition or attribute including body-fat content, size, weight, life stage, sex, coat condition, skin condition, body type, health of the pet, or any combination thereof.

In yet another aspect of the embodiment, the assessment includes an exercise test. For example, the exercise test may be a test that enables a determination of a heart rate of the pet during a period of activity and a heart rate during a period of inactivity. In another example, the exercise test may be a test that enables the determination of a level of exercise the pet can endure.

In still another aspect of the embodiment, the diet regimen may include a recommended daily calorie intake for the pet and/or may include a recommended quantity of specific foods and treats.

In another aspect of the embodiment, the at least one option may include an option for one or more of: a natural (AAFCO) product, an organic product, a brand type, a flavor type, a budget level, a nutritional content, a texture type, a type of filler, a vegetarian product, and a food format.

In yet another aspect of the embodiment, the at least one option may include any combination of: food, treats, supplements, and pharmaceuticals. For example, the food may be wet food, dry food, or a combination thereof.

In a further aspect of the embodiment, the assessment is performed using a computerized survey completed by a veterinarian, by a trained consultant, or by a pet owner. The computerized survey may be obtained from a computer-readable storage medium or a server accessible via the Internet. For example, the computer-readable storage medium may be a CD or a DVD.

In still another aspect of the embodiment, the method includes performing a plurality of assessments of the pet over a period of time during which the pet is on the diet regimen, and refining the diet regimen based on the plurality of assessments.

Optionally, together with the diet regimens of the above-mentioned embodiments, a recommended lifestyle regimen may be provided for the pet, which suggests changes to the pet's exercise routine and suggests how to combine the recommended lifestyle regimen with the recommended diet regimen.

In another option, the diet regimens of the above-mentioned embodiments may include bundled combinations of food products. A bundled combination includes, for example, any two or more of: pet food(s) (wet and/or dry), pet treats, pet supplements (e.g., vitamins and/or herbal products, etc.), and pharmaceuticals. The items in the bundled combination are determined to be best suited for the pet's well-being based on the assessment and based on the feeding preferences and, optionally, any budget considerations the owner may have.

### BRIEF DESCRIPTION OF THE DRAWINGS

The features and advantages of the present invention will become more apparent from the detailed description set forth below when considered in conjunction with the attached drawing.

FIG. 1 schematically illustrates a system diagram of an exemplary assessment system used to implement an embodiment of the present invention.

FIG. 2 illustrates a block diagram of an exemplary method according to the present invention.

### DETAILED DESCRIPTION OF THE INVENTION

The present invention is directed to a method and a system for promoting the longevity and healthy vitality of a pet through a recommended diet regimen. The pet may be a purebred (i.e., with only one breed in its background) or a mixed-breed pet (i.e., have a mixture of breeds within their genetic background). The recommended diet regimen is determined by assessing the pet's condition using a predetermined assessment scheme and taking into consideration the breed type of the pet. The assessment may take into consideration the lifestyle of the pet (e.g., amount and type of exercise, schedule of physical activity, whether confined to indoors only or has outdoor/outdoor exposure, etc.). The assessment may further take into consideration the pet feeding preferences of the pet's owner (e.g., frequency, type(s) of food, type(s) of snacks, type(s) of supplements, prescribed pharmaceuticals, etc.). For example, the recommended diet regimen is determined by using an analysis procedure or algorithm established for the assessment scheme, which takes into consideration the lifestyle of the pet, the pet feeding preferences of the pet's owner, the pet's breed type(s), genetic predisposition to diseases or disorders, size, weight, body-fat content, coat condition, skin condition, body shape, life stage, health, sex, and other physical characteristics. The assessment is conducted with the pet and the pet's owner to determine what the current feeding habits of the pet are (e.g., quantity of food, frequency of feeding, type(s) of food (wet and/or dry), preferred flavors, etc.).

Preferably, the assessment is conducted by a nutrition consultant or a veterinarian who uses information about the breed type of the pet, genetic predisposition to diseases or disorders, a measured body-fat content of the pet, and the pet's weight to determine the condition of the pet. For example, an algorithm may be used to determine the range of products (food (wet and/or dry), treats, supplements, pharmaceuticals) best suited for improving or managing the pet's wellness and health, and promoting a longer life span and a healthier vitality. Optionally, the assessment includes an exercise test, such as a cardio test (e.g., a treadmill test), for example. The exercise test enables a determination of a heart rate of the pet during a period of activity and a heart rate during a period of inactivity. The exercise test also enables a determination of a level of exercise the pet can endure.

The breed type of the pet may be determined by knowing the precise breeding background of the pet. For example, the breed type of a dog may be determined by knowing the ancestry or pedigree of the dog, for example, the breed type of a dog may be determined by knowing the breeds of the parents and/or grandparents of the dog. An estimation of a pet's breed can also be made by examining the physical attributes of the pet. For example, an estimation of a dog's breed may be determined by examining, among others, the color, size, muzzle shape, ear shape, body shape, and tail style of the dog. However, a large proportion of pets are obtained without knowledge of the pet's parentage, or whether these pets are purebred or of a mixed-breed. Therefore, it may be difficult to determine their breed types through a simple examination of their physical attributes alone.

According to an aspect of the invention, the breeds contributing to a mixed-breed pet's background is determined using a DNA-based mixed-breed test. For example, the Wisdom Panel™ MX Mixed Breed Analysis test is a DNA-based mixed-breed test for dogs. The Wisdom Panel™ MX test identifies more than 130 American Kennel Club-recognized breeds that may be present in mixed-breed dogs and therefore, a dog's breed mix can be determined. U.S. Patent Application Publications Nos. 2006/0147962, 2006/0008815, and 2006/0235625, all of which are incorporated by reference herein, provide methods for determining the breed(s) of a dog based on the detection of single nucleotide polymorphisms in the dog. It should be understood that other DNA-based mixed-breed tests for pets may be used.

According to another aspect of the invention, the DNA-based test identifies at lease one dominant or major breed type of a pet. According to another aspect of the invention, the DNA-based test identifies at least one subordinate or minor breed type of the pet. According to yet another aspect of the invention, the DNA-based test identifies a combination of at least one dominant or major breed type of the pet and at least one subordinate or minor breed type of the pet. For example, if a dog is one-half Great Dane, one-quarter German Shepherd, and one-quarter Siberian Husky, the DNA-based test will indicate that the dominant or major breed type of the dog is Great Dane, while the subordinate or minor breeds type of the dog are German Shepherd and Siberian Husky.

It is well-known that different breed types of pets have different nutritional requirements. For example, Boxers are very active, and tend to have a higher percentage of muscle mass than other dogs of similar size; their high level of activity results in added stress to muscles and joints. Accordingly, a diet designed more specifically for Boxers contains a higher energy source (fat) and glucosamine, chondroitin, and omega fatty acids to promote joint health and reduce inflammation. On the other hand, Shih Tzus, for example, are well-known for their hair coat. Maintenance of such a coat requires a high level of the daily protein requirement. Accordingly, a diet designed more specifically for Shih Tzus contains a higher level of protein along with vitamin A and chelated zinc to produce shiny hair and reduce flakiness of the skin, relative to conventional diets.

According to an aspect of the invention, the recommended diet regimen is tailored to benefit the dominant or major breed of the pet. As noted above, different types of breeds have different dietary requirements and therefore, different types of diets are designed for different types of breeds. Accordingly, for example, if a DNA-based test shows that the dominant or major breed of a dog is Great Dane, then at least part of the recommended diet regimen will be specifically designed for Great Danes.

According to another aspect of the invention, the recommended diet regimen is tailored to benefit the subordinate or minor breed of the pet, such that at least part of the recommended diet regimen will be specifically designed for the subordinate or minor breed.

According to yet another aspect of the invention, the recommended diet regimen includes a plurality of dietary items. The plurality of dietary items include at least a first dietary item tailored to benefit the dominant or major breed of the pet and a second dietary item tailored to benefit the subordinate or minor breed of the pet. For example, if a DNA-based test shows that the dominant or major breed of a dog is Labrador and the subordinate or minor breed of the dog is Shih Tzu, then the recommended diet regimen includes a dry food portion that is tailored for Labradors and a wet food portion that is tailored for Shih Tzus. As another example, if a DNA-based test shows that a dog is 40% Great Dane, 40% German Shepherd, 10% Labrador and 10% Golden Retriever, then the recommended diet regimen may include a dry food portion that is tailored for Labradors and Great Danes, a wet food portion that is tailored to German Shepherds, and treats that are tailored for Golden Retrievers.

A genetic predisposition to a disease or disorder may be predicted by examining any diseases or disorders that may be present in any of the breeds of the pet. If one of the breeds of the pet is known to be prone to have an eye condition, then the pet may have a genetic predisposition to such eye condition. For example, English Springer Spaniels are known to be prone to a congenital heart defect known as ventricular septal defect; many breeds of dog, for example, English Cockers, Labrador Retrievers, Chesapeake Bay Retrievers, and Portuguese Water Dogs, are known to be prone to progressive rod-cone degeneration (a form of progressive retinal atrophy or PRA); Deerhounds are known to be prone to osteosarcoma (bone cancer); Golden Retrievers, Great Danes, Beagles, Borzois, Shetland Sheepdogs, American Cocker Spaniels, Labrador Retrievers, Rottweilers, Boxers, Doberman Pinschers, German Shepherds, Akitas, Old English Sheepdogs, and Irish Setters are known to be prone to a thyroid disorder known as autoimmune lymphocytic thyroiditis; and Boxers are known to be prone to certain heart diseases such as dilated cardiomyopathy or subaortic stenosis.

Alternatively, and according to an aspect of the invention, the genetic predisposition to a disease or disorder may be determined by examining the genetic marker associated with the disease or disorder. A genetic marker is a segment of DNA with an identifiable physical location on a chromosome. The segment of DNA is associated with a particular gene or trait and its inheritance can be followed. A marker can be a gene, or it can be some section of DNA with no known function. Because DNA segments that lie near each other on a chromosome tend to be inherited together, markers are often used as indirect ways of tracking the inheritance pattern of a gene or trait whose approximate location is known. Genetic markers associated with certain diseases or disorders can be detected in, for example, the blood or other bodily fluids, a tissue sample, stool, urine, hair, or saliva, and used to determine whether a pet is at risk for developing a disease or disorder.

To date, the locations of numerous genes associated with mammalian diseases/disorders are known. Genetic markers associated with these genes have also been identified. For example, genetic markers for different diseases or disorders in human are well-known in the art. Methods for identifying genetic markers are also well-known in the art. Similarly, genetic markers for different diseases or disorders in pets may be identified. At least one set of genetic markers in canines has been identified. For example, a set of genetic markers that usually indicate the presence of the gene mutation that causes progressive rod-cone degeneration in canines is known. International Patent Publication No. WO 2007/031792, which is incorporated by reference herein, provides a method for determining susceptibility to periodontitis in a Shih Tzu dog, a Yorkshire dog, or a dog of a breed that is genetically related to the Shih Tzu or Yorkshire Terrier breeds based on single nucleotide polymorphisms that are associated with susceptibility to periodontitis in the dog. Therefore, using these genetic markers, genetic predisposition to one or more diseases or disorders may be determined. Examples of genetic diseases/disorders that may be determined from genetic markers include progressive retinal atrophy (PRA), osteosarcoma (bone cancer), gastric dilatation-volvulus (GDV), congenital heart defects, congenital eye diseases, development of food allergies, thyroid conditions, and blood conditions (e.g., von Willebrand disease and hemophilia). Other examples of genetic diseases or disorders may be found in U.S. Pat. No. 7,029,441, which is incorporated by reference herein.

According to an aspect of the invention, the genetic predisposition to a disease or disorder is taken into account when assessing the condition of the pet, and subsequently, a recommended diet is determined that functions to prevent such a disease or enhance an organ that may be affected by the disease. For example, if a dog has a genetic predisposition to PRA, a diet that provides nourishment for the eyes is recommended. Of course, it is understood that the present invention is not limited to the above examples.

Different types of foods may provide nourishment for different organs. Additionally, these different types of foods can be used to manage, delay manifestation, treat, or reduce effects associated with a disease or disorder. For example, the genetic predisposition for the development of food allergies in pets is well-known. Symptoms of food allergies vary widely, however, common symptoms include itching and scratching, swelling, vomiting, weight loss, diarrhea, hair loss, skin lesions, dull coat, and chronic ear infections. One way to alleviate food allergies is to feed the pet lamb-based food; lamb-based diets are uncommon and thus the pet is less likely to be sensitized to the diet. Another way to alleviate food allergies involves food that is intrinsically hypoallergenic, nutritionally-balanced and that when prepared by hydrolysis of food proteins, has a molecular weight range that does not cause diarrhea or other food allergy problems in the pet and that reduces the likelihood of an allergic response. Hypoallergenic pet food may be produced from a proteinaceous component that has undergone sufficient hydrolysis.

Other types of foods known to provide different health benefits or to treat or reduce effects associated with diseases or disorders include, for example, sunflower oil, flaxseed oil, and vitamin E for improving skin and coat health and treating diseases/disorders related thereto; borage oil, flaxseed oil, and green lip mussel extract for improving joint health and treating diseases/disorders related thereto; flaxseed oil, vitamin E, fructooligosaccharide, and chicory for improving digestive health and treating diseases/disorders related thereto. Alternatively, pet diet fortified with glucosamine and chondroitin sulfate in conjunction with gamma linoleic acid may be used to improve and treat diseases/disorders related to joint health. Further, a skin condition that involves itchy and scratchy or flaky skin may be treated with a diet fortified with fatty acids. Furthermore, pet diet fortified with sodium acid pyrophosphate may be used to improve and treat diseases/disorders related to dental and skeletal health. U.S. Pat. No. 6,669,975, which is incorporated by reference herein, provides examples of functional ingredients that could be added to pet food in order to provide different health benefits.

According to an aspect of the invention, the recommended diet regimen is determined by using an analysis procedure or algorithm established for the assessment scheme, which takes into consideration, among others, the skin condition and the body shape of the pet. For example, dogs with wrinkled skin are known to have a higher risk of developing skin issues. Accordingly, a diet that provides nourishment for the skin is recommended. As another example, dogs with different body shapes or conformation have different dietary needs. A dog that is considered to be athletic (e.g., has a body mass index less than 90 kg/m² and an energy requirement of more than 120×(kilograms of ideal body weight)^{0.75} kilocalories per day)) has a different dietary need than a dog that is considered non-athletic (e.g., has a body mass index greater than 90 kg/m² and an energy requirement of less than 120×(kilograms of ideal body weight)^{0.75} kilocalories per day)). For an athletic dog, a diet comprising food ingredients that have a ratio of energy from protein to energy from fat that is less than 0.80 would be recommended. Meanwhile, for a non-athletic dog, a diet comprising food ingredients that have a ratio of energy from protein to energy from fat that is greater than 0.80 would be recommended.

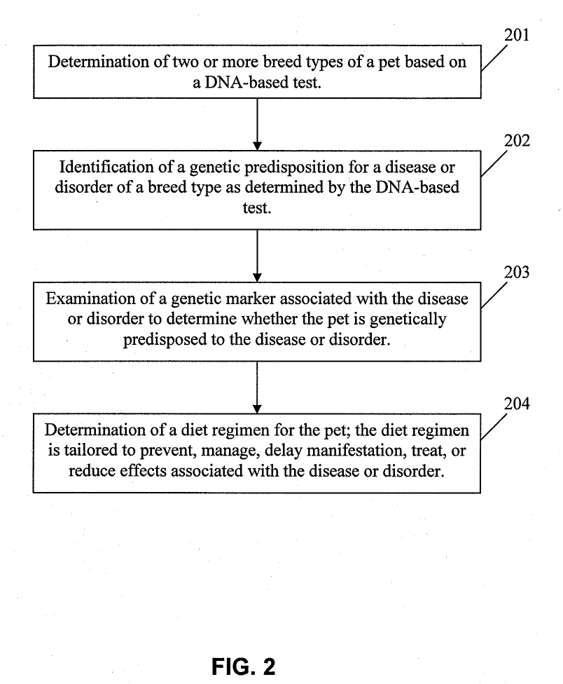

In one embodiment of the invention, a method is provided that promotes longevity and a healthy vitality of a mixed-breed pet, as shown in FIG. 2. The method includes a determination of two or more breed types of the pet, based on a DNA-based test (step 201), an identification of a genetic predisposition for a disease or disorder of a breed type determined based on the DNA-based test (step 202), and an examination of a genetic marker associated with the disease or disorder to determine whether the specific pet is in fact genetically predisposed to the disease or disorder (step 203). The fact that a breed type of a pet is generally known to be prone to a certain disease or disorder does not necessarily mean that the specific pet is genetically predisposed to the disease or disorder. An examination of the genetic marker associated with the disease or disorder can be used to confirm whether the pet is in fact genetically predisposed to the disease or disorder. Once these steps have been performed, a recommended diet regimen for the pet may be determined that is tailored to prevent, manage, delay manifestation, treat, or reduce effects associated with the disease or disorder (if any) (step 204).

According to an aspect of the invention, the pet owner is provided with at least one option for meeting the recommended diet regimen. Optionally, a plurality of options may be provided that take into account the feeding preferences of the owner and enables the owner to diversify or vary the types of food given to the pet. For example, different flavors, shapes, moisture-content amounts, etc., are represented in the plurality of options. Additionally, the plurality of options includes different types of healthy treats for the pet.

Optionally, together with the recommended diet regimen, a recommended lifestyle regimen is provided for the pet, which suggests changes to the pet's exercise routine and suggests how to combine the recommended lifestyle regimen with the recommended diet regimen.

As noted above, optionally, a plurality of options may be provided for the recommended diet regimen, which includes individual food products as well as bundled combinations of food products. A bundled combination includes, for example, any two or more of: pet food(s) (wet and/or dry), pet treats, pet supplements (e.g., vitamins and/or herbal products, etc.), and pharmaceuticals. The items in the bundled combination are determined to be best suited for the pet's well-being based on the assessment and based on the feeding preferences and, optionally, any budget considerations the owner may have. In addition, the plurality of options allows the pet owner to choose from different flavors, shapes, textures, moisture contents, etc., therefore, the pet owner generally will feel that she is doing the best she can for her pet to be happy and healthy and live a long life.

Optionally, a series of assessments may be performed on the pet over a period of time to determine how well the pet is performing under the recommended diet regimen. This enables the recommended diet regimen to be “tweaked” or adjusted in accordance with observations made in the second and subsequent assessments.

The assessment may be performed in a veterinarian's office or in an area of a pet-food market. Preferably, a trained nutrition consultant or a veterinarian performs the assessment. For a pet of a known breed, information can be obtained regarding standard ranges of body-fat content and weight for that breed. As part of the assessment, the pet's characteristics are compared with the standard body-fat and weight ranges as well as other information known about that breed. Optionally, the pet's body fat and weight may be directly measured. Using the weight and body fat information in combination with activity levels (either by owner estimation or through direct measurement of activity level), a determination can be made of the level of exercise the pet can start at in order to, for example, promote weight reduction or improve the pet's health. As mentioned above, a series of assessments may be performed over a period time, which allows the level of exercise recommended for the pet to be modified in accordance with improvements or other changes to the pet's condition.

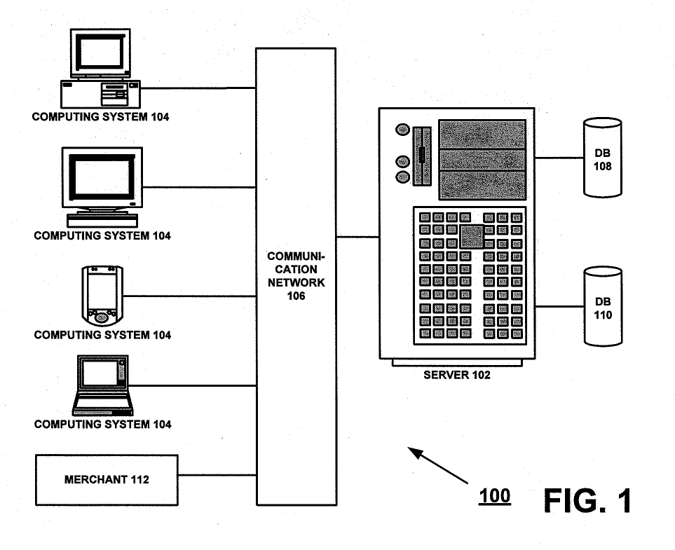

FIG. 1 shows a schematic system diagram of an exemplary assessment system 100, used to implement or practice one or more embodiments of the present invention. System 100 includes a server 102 interconnected with one or more computing systems 104 via a communication network 106. Server 102 has access to a plurality of databases in/from which information is stored/retrieved, such as an assessment-results database 108, which identifies each pet for which an assessment has been performed and stores the assessment results for the pets, and a recommended-diets database 110, which stores a plurality of diet plans each corresponding to an assessment result, for example. Communication network 106 may be the Internet, a public switched telephone network (PSTN), or any other means of communication between server 102 and computing system(s) 104, whether wired or wireless. Computing system 104 may be used by a veterinarian or a trained assessment consultant/associate to communicate with server 102, and may be a personal computer, a workstation, a mainframe computer, a kiosk, a personal digital assistant, or any other digital device able to perform data communication with server 102. Server 102 is programmed to provide an interactive assessment survey to computing system 104, to analyze inputted survey answers, and to provide recommendations according to analysis results.

The assessment survey may be downloaded from server 102 to computing system 104, such that the assessment survey is resident on a hard drive of computing system 104. Optionally, the assessment survey may be loaded in a memory of computing system 104 via a computer-readable storage medium (e.g., a DVD, a CD, etc.) without the need to access server 102.

The assessment survey enables a current condition of the pet to be assessed, based on answers provided to queries in the survey. Additionally, the survey obtains information on feeding preferences of the pet's owner, including preferred: frequency of feedings (e.g., once daily; five-times per day; wet food twice daily and dry food once daily; etc.), type(s) of food (e.g., wet food, dry food, only natural food(s), a combination of natural and regular foods, etc.), use of particular products for treats (e.g., use of oral-care dry food as treats, etc.), for example. The term “natural,” as used herein, generally refers to foods that have by-product specifications established by the AAFCO, as will be appreciated by persons skilled in the art. A recommended diet is determined based on the assessment. (As used herein, the term “diet” includes any combination of foods (wet and/or dry), treats, supplements, and pharmaceuticals). The pet's owner enrolls the pet in a desired feeding plan based on the recommended diet and, optionally, the owner's pet-maintenance budget. For example, for a modest-budget feeding plan (e.g., a “Bronze Plan”), the recommended diet includes a combination of standard foods/treats/supplements/pharmaceuticals chosen to improve or manage the pet's condition; for a high-budget feeding plan (e.g., a “Platinum Plan”), the recommended diet includes premium foods/treats/supplements/pharmaceuticals chosen to improve or manage the pet's condition. As will be appreciated by persons of skill in the art, feeding plans for other budget levels also may be available and within the scope of the present invention (e.g., a “Gold Plan,” a “Silver Plan,” etc.).

As used herein, the term “foods/treats/supplements/pharmaceuticals” is intended to indicate any combination of two or more of the four categories of feeding-plan products (i.e., foods (wet and/or dry) and treats; foods, treats, and pharmaceuticals; foods, treats, and supplements; etc.); the combination is not required to include all four categories of products. Preferably, the combination includes food (wet and/or dry) and at least one other category of feeding-plan products.

The pet's owner enrolls in the feeding plan through a merchant 112, such as a supermarket for pet-related products. The owner then obtains the plan's foods/treats/supplements/pharmaceuticals at a convenient predetermined location, such as the merchant's store. The owner is given an incentive by merchant 112 to enroll in the plan and to make repeat purchases of the plan's foods/treats/supplements/pharmaceuticals from the merchant's store. For example, the incentive may be that the total cost to the owner of the food/treats/supplements if purchased as a bundle under the plan would be less than the cumulative cost to the owner of the food/treats/supplements/pharmaceuticals if purchased separately (e.g., the fixed cost for a purchase under the plan gives the owner a discount of 30%, for example, for the combined purchase of the foods/treats/supplements/pharmaceuticals); or the incentive may be a discount card that enables every fifth bundled purchase under the plan to receive a discount of, for example, 50%. In another example, the incentive may be to provide the owner with a gift for signing on to purchase periodic bundles for a term of, for example, one year. Other types of loyalty incentives are also contemplated.

As will be appreciated by persons of skill in the art, and as discussed above, pharmaceuticals need not be included in the plan, especially if merchant 112 is not licensed to dispense pharmaceuticals. However, if the plan is purchased from a veterinarian who is licensed to dispense pharmaceuticals, then the plan may include pharmaceuticals.

Each plan provides, on a periodic basis (e.g., bi-weekly, monthly, etc.), sufficient foods/treats/supplements/pharmaceuticals for the period. For example, a monthly Bronze Plan for a cat may provide a month's supply of standard dry and/or wet cat food, a container of about 60 standard treats (suitable for about 2 treats per day), a month's supply of standard vitamins and/or other types of supplements, and if necessary a month's supply of generic pharmaceuticals for treating a heart condition. Similarly, for a monthly Platinum Plan, a month's supply of premium dry and/or wet cat food, a month's supply of premium treats, a month's supply of premium supplements, and a month's supply of name-brand pharmaceuticals may be included in the plan. The types of foods/treats/supplements/pharmaceuticals provided depends on the results of the assessment for the cat (i.e., the recommended diet, which takes into account, among others, the feeding preferences of the pet's owner). Therefore, if the cat is assessed to have a dull coat, then suitable vitamins and/or other types of supplements may be included in the plan; if the cat is advanced in age, then the plan's food may be food for senior cats; if the owner prefers only natural foods, then the plan may include only natural foods; if the owner prefers natural and/or indulgent foods, then the plan may include one or both of natural foods and indulgent foods; and if the cat requires pharmaceuticals, then the plan may include suitable pharmaceuticals.

If the assessment determines that the feeding preferences of the pet's owner is to feed the pet small quantities of wet food several times (e.g., five or more times) per day, then the feeding plan may include small single-serving wet-food pouches that avoids the need for the owner to store opened cans of partially used food. This feature removes the uncertainty the owner may have about the appropriate amount of food to feed the pet, especially when the pet is fed several times per day, and also removes the uncertainty of whether the proper food is being given to the pet. In addition, single-serve pet food allows a pet owner to feed freshly opened food to the pet every time without having to worry about spoilage or refrigeration of any leftover.

According to an embodiment of the invention, the assessment is performed based on a computerized survey, in which a veterinarian uses a computer to input answers to the survey. The answers automatically are analyzed according to an algorithm designed to assess the health and well-being of the pet under observation. The computer then outputs the recommended diet for the pet. Optionally, instead of a veterinarian, a person trained to understand the survey and the process of assessing a pet's condition may perform the assessment, such as a consultant/associate located at the merchant's store or at the veterinarian's office.

The computerized assessment survey may be in the form of an interactive program stored locally in a memory unit of computing system 104 and executed by a microprocessor of computing system 104. That is, the program may be locally installed in a computer used by the veterinarian or the consultant/associate without requiring that the computer be in communication with server 102. This allows the veterinarian or the consultant/associate to perform “mobile” pet assessments using, for example, a laptop at any desired location (e.g., a shopping mall), which may not provide an easy way for the laptop to connect to a communication network. If desired, results from such mobile assessments may later be uploaded to server 102 so that they may be stored in database 108. As will be appreciated by persons of skill in the art, when the program is locally installed in computers used by veterinarians or consultants/associates, a plurality of diet plans each corresponding to an assessment result is stored locally in the computers in association with the locally-installed program.

Alternatively, as mentioned above, the interactive program may be stored off-site in a memory unit accessible by server 102 and shared (accessible) by other computing systems 104, and is accessible by each computing system 104 through communication network 106. The program provides an electronic user interface that queries the veterinarian or the consultant/associate to provide responses to survey questions about the pet being examined. Once the responses have been entered, via computing system 104, the program uses the responses in an algorithm to determine the recommended diet or to provide suggestions for various diets, which are discussed with the pet's owner before a plan is chosen.

Preferably, the veterinarian or the consultant/associate encourages the pet's owner to enroll the pet in a feeding plan by informing the owner of the benefits to the pet of a combined regimen of the plan's foods/treats/supplements/pharmaceuticals, and by informing the owner of the savings the owner would be entitled to if the plan's foods/treats/supplements/pharmaceuticals are purchased together in a bundle from merchant 112 than if purchase separately. According to one option, computing system 104 transmits information on the pet and the pet's recommended diet to a computer at the merchant's store for the convenience of the pet's owner. Then, when the owner goes to the merchant's store, a feeding-plan consultant discusses various feeding-plan options (e.g., Bronze Plan, Platinum Plan, etc.) with the owner and determines the desired feeding plan for the pet. The owner then purchases the plan's foods/treats/supplements/pharmaceuticals on a periodic basis or as needed from the merchant's store. According to another option, computing system 104 transmits information on the pet, the pet's recommended diet, and the desired feeding plan to a computer at the merchant's store for the convenience of the pet's owner. Then, the owner can purchase the plan's foods/treats/supplements/pharmaceuticals on a periodic basis or as needed from the merchant's store.

According to another embodiment of the invention, the assessment is performed manually and the veterinarian uses a “recommended-diet notepad” similar to a physician's prescription pad to note the pet's recommended diet and to direct the pet's owner to the merchant's store to determine the desired feeding plan. The notepad may include pre-printed information about the different feeding plans available as well as information about incentives for enrolling in a plan, as discussed above.

According to yet another embodiment of the invention, various aspects of the computerized survey and the manual assessment may be combined.

According to a further embodiment of the invention, the assessment is performed by the pet's owner according to a predetermined computerized assessment survey, in which the owner uses computing system 104 to input answers to the survey. That is, involvement by a veterinarian or a consultant/associate is not necessary. The answers inputted by the owner automatically are analyzed according to an algorithm designed to assess the health and well-being of the pet being assessed. The computer then outputs the recommended diet for the pet.

The assessment survey may be in the form of an interactive program stored in a memory unit accessible by server 102 and shared (accessible) by pet owners, veterinarians, pet caretakers, and the like, through computing systems 104 communicating over communication network 106. The program provides an electronic user interface that queries the pet owner to provide responses to survey questions about the pet being assessed. Once the responses have been entered, the program uses the responses in an algorithm to determine the recommended diet.

Preferably, the program presents the pet's owner with information encouraging enrollment of the pet in a feeding plan offered by the merchant by informing the owner of the benefits to the pet of a combined regimen of the plan's foods/treats/supplements/pharmaceuticals, and by informing the owner of the savings the owner would be entitled to if the plan's food/treats/supplements are purchased together from merchant 112 than if purchase separately. Then, when the owner goes to the merchant's store, a feeding-plan consultant discusses various feeding-plan options (e.g., Bronze Plan, Platinum Plan, etc.) with the owner and determines the desired feeding plan for the pet. The owner then purchases the plan's foods/treats/supplements on a periodic basis or as needed, as discussed above.

According to an aspect of the embodiment, a kiosk/booth at the merchant's store or at the veterinarian's office may be used by the pet's owner to complete the survey to determine the recommended diet for the pet. This way, the owner conveniently may determine the recommended diet, decide on a desired feeding plan, and purchase foods/treats/supplements/pharmaceuticals for the feeding plan all at the same location. Preferably, the kiosk/booth is equipped with a computer or other type of communication device (e.g., computing system 104) for accessing server 102, which controls an interactive assessment program for a plurality of kiosks/booths.

According to still another embodiment of the invention, assessments of the pet are made over a period of time to determine the efficacy of the recommended diet in maintaining or improving the pet's condition. The assessments are stored in database 110 and used to refine the program's algorithm for determining future recommended diets.

The various embodiments of the present invention described above have been presented by way of example and not limitation. It will be apparent to persons skilled in the relevant arts that various changes in form and detail can be made therein without departing from the spirit and scope of the present invention. Thus, the present invention should not be limited by any of the above-described exemplary embodiments, but should be defined only in accordance with the following claims and their equivalents. It is also to be understood that the steps and processes recited in the claims need not be performed in the order presented.

In addition, it should be understood that the attached drawing, which highlights the functionality and advantages of the present invention, are presented as an illustrative example. The system arrangement of the present invention is sufficiently flexible and configurable, such that it may be utilized and configured in ways other than that shown in the drawing.

What is claimed is:

1. A method of promoting longevity and a healthy vitality of a pet, comprising:
performing an assessment of a condition of the pet, wherein the assessment takes into consideration a breed type of the pet determined by a DNA-based test;
determining a diet regimen for managing the condition of the pet, based on the assessment; and
providing at least one option for satisfying the diet regimen.

2. The method of claim 1, wherein the assessment includes determining a genetic predisposition of the pet for a disease or disorder.

3. The method of claim 2, wherein the genetic predisposition of the pet to the disease or disorder is determined by examining a genetic marker associated with the disease or disorder.

4. The method of claim 3, wherein the diet regimen is tailored to prevent, manage, delay manifestation, treat, or reduce effects associated with the disease or disorder to which the pet is genetically predisposed.

5. The method of claim 1, wherein the assessment takes into consideration at least one physical condition or attribute of the pet, the physical condition or attribute including body-fat content, size, weight, life stage, sex, coat condition, skin condition, body type, health of the pet, or any combination thereof.

6. The method of claim 1, further comprising:
performing a plurality of assessments of the pet over a period of time during which the pet is on the diet regimen; and
refining the diet regimen based on the plurality of assessments.

7. A method of promoting longevity and a healthy vitality of a mixed-breed pet, comprising:
determining two or more breed types of the pet, based on a DNA-based test;
identifying a genetic predisposition for a disease or disorder of a breed type determined based on the DNA-based test; and
examining a genetic marker associated with the disease or disorder to determine whether the pet is genetically predisposed to the disease or disorder,
wherein the genetic marker is obtained from a bodily fluid or a tissue sample of the pet.

8. The method of claim 7, further comprising:
determining a diet regimen for the pet; and
providing at least one option for satisfying the diet regimen.

9. The method of claim 7, further comprising determining a diet regimen for the pet, wherein the diet regimen is tailored to prevent, manage, delay manifestation, treat, or reduce effects associated with the disease or disorder.

10. The method of claim 9, further comprising:
performing a plurality of assessments of a condition of the pet over a period of time during which the pet is on the diet regimen; and
refining the diet regimen based on the plurality of assessments.

11. The method of claim 7, wherein the DNA-based test identifies at least one dominant breed type of the pet or at least one minor breed type of the pet or a combination thereof.

12. The method of claim 11, further comprising determining a diet regimen for the pet, wherein the diet regimen is tailored benefit a dominant breed of the pet.

13. The method of claim 11, further comprising determining a diet regimen for the pet, wherein the diet regimen is tailored benefit a minor breed of the pet.

14. A method of promoting longevity and a healthy vitality of a mixed-breed pet, comprising:
performing an assessment of a condition of the pet, wherein the assessment takes into consideration at least one breed type of the pet determined by a DNA-based test;
determining a diet regimen for managing the condition of the pet, based on the assessment; and
providing at least one option for satisfying the diet regimen.

15. The method of claim 14, wherein the DNA-based test identifies at least one dominant breed type of the pet or at least one minor breed type of the pet or a combination thereof.

16. The method of claim 15, wherein the diet regimen is tailored to benefit a dominant breed of the pet.

17. The method of claim 15, wherein the diet regimen is tailored to benefit a minor breed of the pet.

18. The method of claim 14, wherein the assessment includes determining a genetic predisposition of the pet for a disease or disorder.

19. The method of claim 18, wherein the diet regimen is tailored to prevent, manage, delay manifestation, treat, or reduce effects associated with the disease or disorder to which the pet is genetically predisposed.

20. The method of claim 14, wherein the assessment takes into consideration at least one physical condition or attribute of the pet, the physical condition or attribute including body-fat content, size, weight, life stage, sex, coat condition, skin condition, body type, health of the pet, or any combination thereof.

21. The method of claim 14, further comprising:
performing a plurality of assessments of the pet over a period of time during which the pet is on the diet regimen; and
refining the diet regimen based on the plurality of assessments.
